(19) United States
(12) Patent Application Publication
(10) Pub. No.: US 2025/0001047 A1
(43) Pub. Date: Jan. 2, 2025

(54) Instrumented Cartridge or Insert for Detecting Infections in Implanted Medical Devices

(71) Applicant: BRIDGEWAY INNOVATIONS, LLC, College Station, TX (US)

(72) Inventors: Tyler Touchet, Cypress, TX (US); Duncan J. Maitland, College Station, TX (US); Landon D. Nash, Park City, UT (US)

(21) Appl. No.: 18/755,887

(22) Filed: Jun. 27, 2024

Related U.S. Application Data
(60) Provisional application No. 63/523,513, filed on Jun. 27, 2023.

(51) Int. Cl. A61L 27/50; A61B 5/00; A61L 27/14; A61L 31/04; A61L 31/18

(52) U.S. Cl. CPC A61L 27/50; A61B 5/4851; A61L 27/14; A61L 31/04; A61L 31/18; A61L 2400/06

(57) ABSTRACT

A embodiment includes a system comprising: (1) a medical implant comprising at least one of a bone anchor, a hip implant, a knee implant, a shoulder implant, an orthopedic plate, or combinations thereof; (2) a sealed housing coupled to the medical implant; (3) a liquid crystal elastomer (LCE) included in the sealed housing; wherein the LCE is configured to transition from a nematic phase to an isotropic phase in response to exposure of the LCE to a temperature above 38 degrees Celsius.

### CROSS-REFERENCE TO RELATED APPLICATION

This application claims priority to U.S. Provisional Patent Application No. 63/523,513 filed on Jun. 27, 2023 and entitled “Instrumented Cartridge or Insert for Detecting Infections in Implanted Medical Devices”, the content of which is hereby incorporated by reference.

### TECHNICAL FIELD

Embodiments of the invention are in the field of medical devices and, in particular, infection detection in medical devices.

### BACKGROUND

Bacteria may adhere to the surface of medical devices, producing biofilms. Device-related infection may result in tissue destruction, systemic dissemination of the pathogen and dysfunction of the device, causing increased morbidity and mortality. These infections are resistant to immune defense mechanisms and are difficult to treat with antimicrobial agents. Removal of the device may be necessary with attendant distress to the patient and cost.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIGS. 1(a)-(b) depict cross sectional views of a helical liquid crystal elastomer (LCE) actuator embodiment. FIGS. 1(c)-(d) address imaging of the embodiment of FIGS. 1(a)-(b).

FIGS. 2(a)-(b) depict cross sectional views of a columnar LCE actuator. FIGS. 2(c)-(d) address imaging of the embodiment of FIGS. 2(a)-(b).

FIGS. 3(a)-(b) depict embodiments of an instrumented infection detection cartridge for orthopedic fixation devices.

FIG. 4 depicts a schematic of an assembly method for a thermal sensor.

FIGS. 5(a)-(b) depict cross sectional views of a helical temperature actuated element actuator. FIGS. 5(c)-(d) address imaging of the embodiment of FIGS. 5(a)-(b).

FIGS. 6(a)-(b) depict cross sectional views of sealed disk-shape housing that encloses a helical LCE actuator. FIGS. 6(c)-(d) address imaging of the embodiment of FIGS. 6(a)-(b).

FIGS. 7(a) and 7(c) depicts a cross-sectional view of sealed temperature sensitive sensors respectively at body temperature and at temperatures above body temperature. FIGS. 7(b) and 7(d) provide top views of the respective embodiments of FIGS. 7(a) and 7(c).

FIG. 8(a) depicts a piece of orthopedic fixation hardware, such as a bracket to use with anchoring screws. FIGS. 8(b)-8(c) show how during implantation, infection detection cartridge embodiments are applied to extra holes not used for fixation.

FIG. 9 shows a schematic of a temperature dependent polymer system embodiment that changes empirical parameters through changes in a secondary radiopaque material.

FIG. 10 shows an embodiment of an infection detection array of radiopaque or echogenic particles embedded in a temperature sensitive polymer matrix.

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

Embodiments concern non-invasive, in situ diagnosis of infections associated with medical implants, including orthopedic implants.

Applicant determined there is a need for clinicians to have an early and accurate indicator of a developing infection after the implantation of a medical device. Orthopedic examples include an artificial joint such as a hip, knee, or shoulder replacement; structural plates for fixation; screws including pedicle and cannulated; spinal cages; orthopedic wedges; or other orthopedic hardware. Embodiments address approaches to provide clinicians with a reliable signal to indicate the early stages of a device infection, which allows proactive intervention before a clinically significant infection is established. Early infection intervention may prevent or lessen patient discomfort and the complete removal and revision of the implant, which are common for severe orthopedic implant infections. Additionally, early detection of infection can reduce the burden of sustained care for the patient, hospital investment, and post-operative protocols. Many embodiments addressed herein enable non-invasive assessment of infection in a way that integrates with existing clinical workflows (i.e. x-ray imaging).

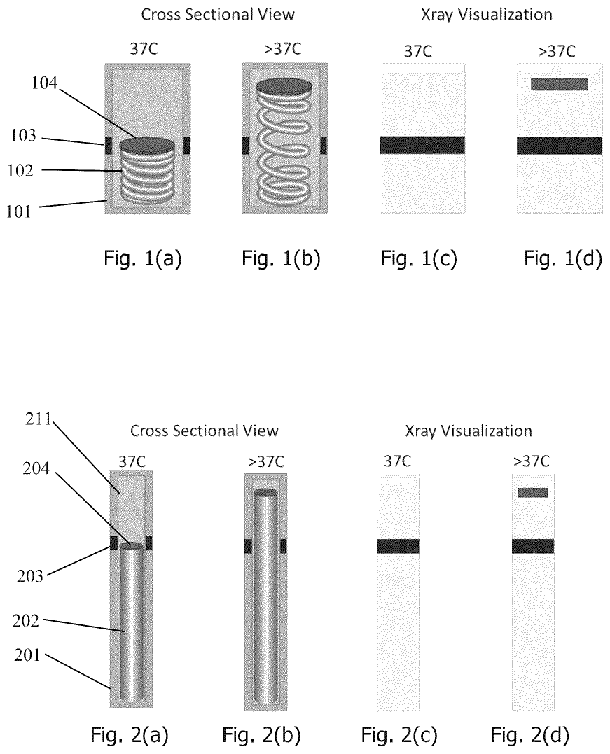

FIGS. 1(a)-(d) depict a sealed tubular housing 101 that houses a helical liquid crystal elastomer (LCE) actuator 102 that is fixably coupled to the housing on one end, and a mobile radiopaque marker 104 fixably coupled to the opposite end of the LCE. The housing also includes a fixed radiopaque feature 103, such as a marker band. At elevated temperatures, the helical LCE actuator changes physical conformation and moves the mobile radiopaque marker on the LCE relative to the fixed radiopaque marker on the housing. This physical conformation change is reversible, and the LCE and mobile marker band return (fully or partially) to the original position (or near the original position) after a decrease in temperature. FIG. 1(a) provides a cross-sectional view of a cartridge assembly at body temperature. FIG. 1(b) provides a cross-sectional view of the cartridge assembly at elevated temperature. FIG. 1(c) provides an x-ray visual of the mobile and static radiopaque markers at body temperature. FIG. 1(d) provides an x-ray visual of the static and mobile radiopaque markers at elevated temperatures.

The sealed tubular housing can be inserted into, for example, features of orthopedic hardware to measure infection related temperature changes. In an embodiment the housing is sealed to prevent fouling the LCE actuation with cellular and biologic debris. The interior volume of the sealed tubular housing can be filled with entrapped gasses (including but not limited to ambient air, carbon dioxide, nitrogen, argon, or combinations thereof) or fluids (including but not limited to water, semi-polar solvents, non-polar solvents, or combinations thereof) to increase thermal conductivity between the housing and the LCE actuator. The housing can be polymeric, or in an ideal embodiment, metallic. Exemplary metal alloys for the tubular housing include stainless steel, cobalt chromium, or titanium. In an embodiment, the housing alloy is matched to the target orthopedic hardware alloy composition to prevent galvanic corrosion between the instrumented cartridge and orthopedic implant.

In an embodiment, the actuating element has a negative coefficient of thermal expansion to compensate for volume increases in the sealed volume due to the heat conducting fluid, and or housing. In an embodiment a fluid filled cartridge can include a small volume of compressible gas to compensate for pressure changes within the sealed cartridge.

FIGS. 2(a)-(d) address a columnar LCE actuator 202/housing 201 embodiment. FIG. 2(a) depicts axial conformation at body temperature and FIG. 2(b) depicts a changed axial conformation at elevated temperatures to thereby move a mobile radiopaque marker 204 distal (up in FIG. 2(b)) relative to a static radiopaque feature 203 on the housing. Under x-ray visualization, FIG. 2(c) illustrates the radiopaque markers are aligned at body temperature and FIG. 2(d) illustrates a detectable gap at elevated temperatures between the markers. In an alternative embodiment, the LCE shortens in conformation at elevated temperature, moving the mobile radiopaque marker proximal to the static radiopaque marker. The radiopaque markers can be any high Z element, with various embodiments using platinum alloys with tungsten and iridium. The mobile radiopaque marker can be a disk, cylindrical hoop (marker band), wire filament, cylindrical coiled filament, and the like. The static marker feature could include, for example, a platinum alloy marker band swaged to the cartridge housing, or a wound cylindrical coiled platinum filament. The LCE cross section can be circular, oval, square, or rectangular to augment the column strength and stiffness of the actuator.

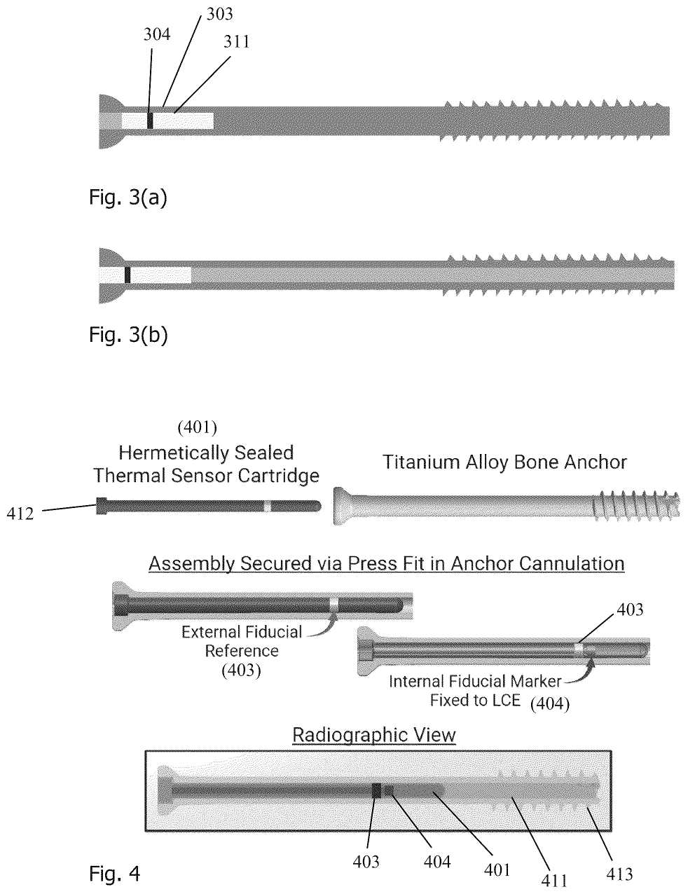

FIGS. 3(a)-(b) depict embodiments of an instrumented infection detection cartridge. FIG. 3(a) illustrates the cartridge as integrated into an orthopedic fixation device. For example, the cartridge may have walls that are monolithic with the device. For example, wall 311 may be milled or otherwise formed within the anchor and may also form a sidewall analogous to sidewall 211 of FIG. 2(a). Radiopaque feature 304 is analogous to features 104, 204 addressed above and may be judged against a static radiopaque feature located at, for example, location 303. FIG. 3(b) illustrates the cartridge as inserted into a void (such as a cannulation or screw head feature) after the fixation device is implanted. Such a cartridge may be entirely separable from the anchor and may take the form of embodiments shown in, for example, FIGS. 1(a) and/or 2(a). The inserted cartridge can fixably couple to the orthopedic fixation device via interference fit, internal threads in the cannulation (counter clockwise or clockwise rotation relative to the torquing direction of the orthopedic screw direction), bayonet locking mechanism, detents, and the like.

FIG. 4 depicts a schematic of an assembly for a thermal sensor with housing 401 having cap 412. The radiographic view (bottom) is at room temperature (25 C). At body temperature (37 C), the mobile marker band 404 would move proximal (to the left and towards the anchor head) to align with the external fiducial marker 403. At elevated temperatures (>37 C) the actuator would move proximally to the external fiducial reference.

In an embodiment, a thermal sensor (similar to the system of FIG. 4) is comprised of at least five components: (1) an LCE ribbon, (2) titanium alloy tubing as an outer body, (3) a titanium alloy cap, and (4, 5) two platinum fiducial marker bands. The titanium alloy screw will be nominally 48 mm long, 4.5 mm in diameter, with a 1.3 mm inner diameter cannula. The sensor will be assembled in a staged process, during which a fiducial marker (platinum band) will be affixed to the LCE at the distal end. The proximal end of the material will be potted via adhesive to a cap (e.g., see cap 412 of FIG. 4) to create the sensing element. The sensing element and outer body will be permanently bonded using biocompatible epoxy (MED-OD2002, Epoxy Technology, Inc.). To prevent unwanted interactions between the LCE and the inner surface of the outer body (e.g., see “inner surface”/wall 411 of “outer body”/screw 413) and increase thermal conductivity, the tube will be filled with non-pyrogenic water and sealed with the same epoxy used to bond the sensing element to the cap. Finally, the external fiducial marker will be placed on the titanium tubing.

In an embodiment, the sensor may be designed such that the sensor either reads a normal or elevated temperature reading. This dichotomous sensor will enable facile integration with current workflows associated with device observation. A 40 mm LCE fiber may generate 2 mm-5 mm of displacement (contraction) on heating 5° C. above physiological conditions. The relative distance between the two platinum markers is a freely changeable design variable. An embodiment may provide a gap that can be observed between the two platinum markers over 2.5° C. of heating occurs. The presence of the imageable gap between fiducial markers when the radiograph is taken normal to the length of the bone will indicate elevated temperature. Moist heat is commonly used in orthopedics to achieve terminal sterilization. Therefore, to remain consistent with current workflows, terminal sterilization may be performed in accordance with AAMI ST79:2017 using the standard parameters of 132° C. for 4 minutes. Sterilized and unsterilized sensors may be tested using the workflow in Sub-aim 2C to elucidate any change to performance caused by sterilization.

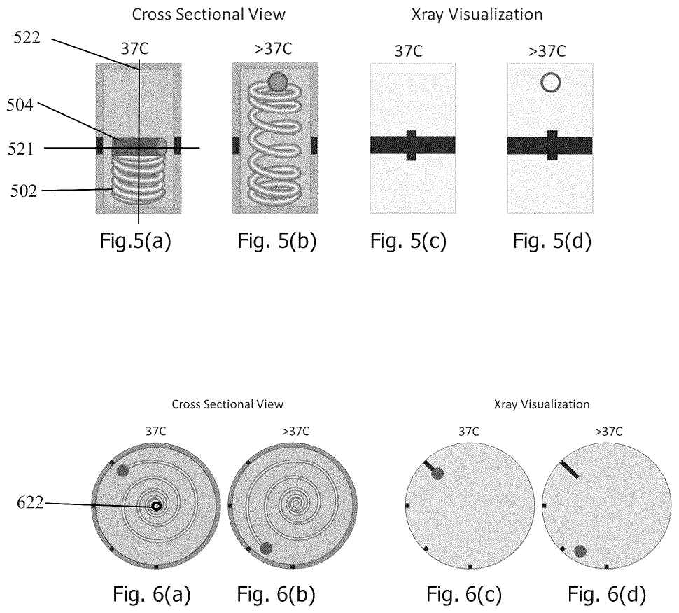

FIGS. 5(a)-(d) include a sealed cylindrical cartridge with a helical temperature actuated element 502. As the helix changes confirmation, a tubular radiopaque marker 504 changes axial position and radial position relative to a static fiducial marker. FIG. 5(a) depicts at body temperature; the mobile marker is axially aligned with the static radiopaque fiducial on the cylindrical housing. The cylindrical marker is aligned with the cross-sectional plane of the figure. FIG. 5(b) depicts at temperatures above body temperature, the helical temperature actuating element has extended and twisted relative to the starting position. The tubular radiopaque element has axially extended relative to the static radiopaque fiducial on the housing, and the tubular element is now orthogonal to the cross-sectional plane 521. FIG. 5(c) shows the x-ray visualization corresponding to FIG. 5(a). FIG. 5(d) shows the Xray visualization corresponding to FIG. 5(b).

FIGS. 6(a)-(d) depict a sealed disk-shape housing that encloses a helical LCE actuator fixably coupled to the housing at the center 622 of the helix and the distal end of the LCE actuator is fixably coupled to a mobile radiopaque marker. As the LCE actuator increases in temperature, the LCE changes physical conformation and radially translates the mobile radiopaque marker relative to static radiopaque features on the disk housing.

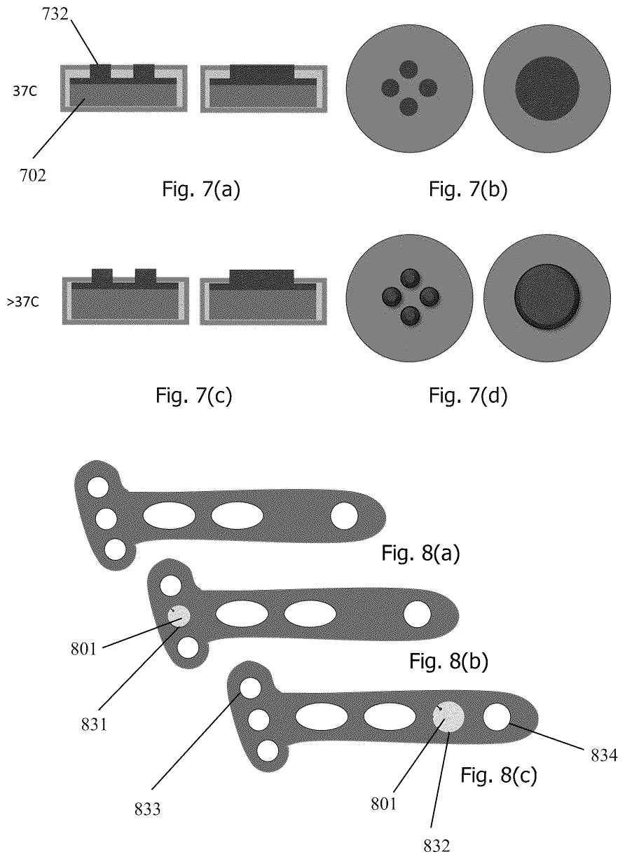

FIG. 7(a) depicts a cross-sectional view of a sealed temperature sensitive sensor at body temperature, where the features on the top face of the cartridge are flush. FIG. 7(b) is a top view of the cartridges shown in FIG. 7(a). FIG. 7(c) depicts a cross-sectional view of a sealed temperature sensitive sensor at temperatures above body temperature, where a temperature sensitive actuator raises features above the surface of the cartridge. These features can be palpated to detect increases in localized implant temperature. Such an embodiment may include, for example, LCE actuator 702.

FIG. 8(a) depicts a piece of orthopedic fixation hardware, such as a bracket to use with anchoring screws. FIG. 8(b) shows how during implantation, one of the instrumented infection detection cartridge embodiments 801 (e.g., see FIG. 6(a)) is applied to an extra hole (831) that is not used for fixation. Other windows (833, 834) are maintained to use with screw fixation. FIG. 8(c) shows where alternatively, the cartridge can be integrated into the device in a designated location 832 at the time of bracket manufacturing.

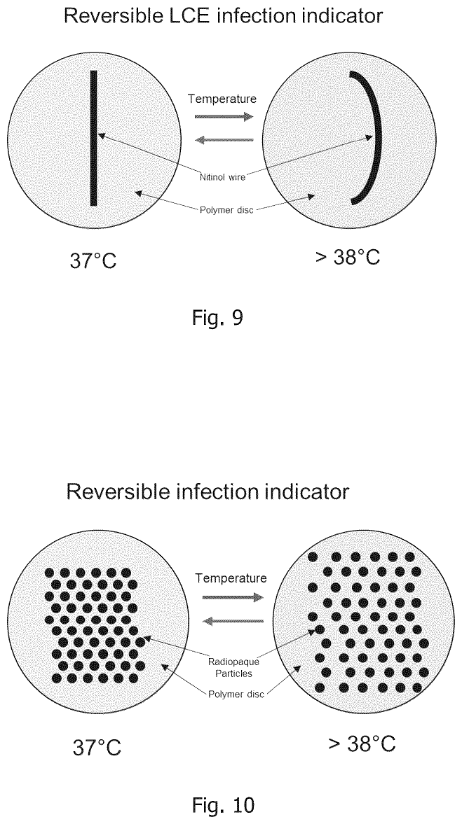

FIG. 9 shows a schematic of a temperature dependent polymer system that changes empirical parameters through changes in a secondary radiopaque material.

FIG. 10 shows an infection detection array of radiopaque or echogenic particles embedded in a temperature sensitive polymer matrix. As temperature changes, the thermal expansion behavior of the polymer alters the spacing of the particles, changing the radiopacity or echogenic properties of the construct. These properties can be measured via fluoroscopy or ultrasound imaging.

With the Figures addressed above, a description of varying embodiments is now addressed. Embodiments include a cartridge consisting of:
1. A body or frame that has two primary functions: holding the sensing and readout elements and a method of attaching the cartridge into the implanted device or stabilizing the device to the implantation site.
2. A sensing element that reacts to physiological parameters to change its physical conformation or signaling properties.
3. A communication element that can be non-invasively read outside the patient's body. The communication element provides a quantitative, semi-quantitative or qualitative measure a physiological parameter or parameters and/or indicator of infection.

An embodiment includes a frame or body that includes all sensing and read-out elements that can be inserted into medical devices before and/or after implantation. Configurations of the frame are cylindrical or disk shaped. Alternative embodiments include the sensing read-out elements in a compliant envelope.

An embodiment includes a frame or device body that includes all sensing and read-out elements where all mobile components are sealed within the device body, preventing fouling from biologic fluids, cellular activity, and scar formation.

An embodiment includes a frame or device body that includes all sensing and read-out elements where at least one of the sensing or read out elements is exposed to the biologic environment to sense physiologic parameters.

An embodiment includes a frame and sensing elements integrated or partially integrated such that some or all of the sensing and read out elements are integrated into the device channel or device itself. For example, the sensing and communication elements are integrated into an orthopedic anchor or plate.

An embodiment includes a sensing element that, with the exposure of different levels of the sensed parameter, converts or transduces the element state into 1D transformation such as a linear or other extension/contraction, up to 4 dimensional changes (i.e. volumetric changes over time), a physical state that can be non-invasively read, or a digital signal.

An embodiment includes non-invasive reading options that include location or opacity changes that can be read with X-ray images, location or density changes that can be read with ultrasound images or ultrasound signals, optical changes that can be read with light intensity and/or phase, optical changes that can be read with phosphorescence or fluorescence lifetimes, and tactile or shape changes that can be read by clinician palpitation or touch through the skin.

An embodiment includes a temperature sensor that converts temperature into the linear translation of radio opaque markers or marker that can be visually read by a human or computer to read the temperature of the cartridge. The temperature scale can be quantitative, semi-quantitative or qualitative (yes/no temperature).

A linear translation in response to physical analytes that moves a readable marker with a translation range between 0.1 mm and 100 mm corresponding to the range of interest in the analyte. For example, an actuator with an initial length of 20 mm that elongates (or shortens) between 0-5 mm indicates a temperature of 37-45 C. In one embodiment, a length of 20 mm indicates 37 C and 25 mm indicates 45 C. In another embodiment, a length of 20 mm indicates 45 C and a 25 mm length indicates 37 C. A second example is an actuator with an initial length of 10 mm that has a shortening range of 0-1 mm over the same temperature range. In the second example, a length of 10 mm indicates 37 C and 9 mm indicates 45 C.

A temperature sensor that is sensitive over the temperature range of 30-50 C or a subset of that temperature range.

An embodiment includes an X-ray readable radiopaque marker that changes location relative to one or more reference (fiducial) radiopaque markers. The reference markers help the clinician read the movement of the infection-indicating marker. Radiopaque markers are comprised of materials with higher radiodensity than other device materials. For example, tungsten, tantalum, platinum, iridium, barium sulfate.

An embodiment includes a sensing cartridge integrated into orthopedic anchors, particularly cannulated anchors, with X-ray readouts of one or more physical analytes. The cartridge can fit in cannulations with luminal diameters of 0.5-10 mm and lengths of 5-200 mm.

An embodiment includes a system capable of measuring or responding to analytes of interest including concentrations of uric acid, pH, d-lactate, a-defensin, C-reactive protein, erythrocyte sedimentation rate, procalcitonin, leukocyte esterase, reactive oxygen species, individually or in combinations of two or more. A temperature sensor or transducer can be used either alone or in combination with one or more of the chemical sensors or chemical transducers.

An embodiment includes a sensor read out by tactile feel by the healthcare provider. In this embodiment, a physiologically sensitive actuator 702 is used to raise physical features 732 on the surface of an implant in response to a biologic response to infection. An example would be a temperature sensitive actuator raising features on the surface of an orthopedic implant in response to an increase in local tissue temperature due to infection. This embodiment is especially useful for orthopedic hardware present superficially under the skin, making it straightforward for clinicians to manually palpate. In one embodiment, the actuator is sealed in a cartridge to prevent fouling of the actuating components. The raised features can be sealed with a flexible, waterproofing membrane such as a silicone film. See, for example, FIGS. 7(a)-(d). Components 702 and 732 may be included in the silicone film.

An embodiment includes a cartridge that is inserted into the internal lumen of a cannulated orthopedic screw after implantation, or integrated into a non-cannulated screw at the time of screw manufacture. See, for example, FIGS. 3(a)-3(b). The detected signal is x-ray imaging and measurement of the distance between a static fiducial radiopaque feature on the body of the cannulated screw or housing of the inserted cartridge, and a second mobile radiopaque marker fixably coupled to a temperature sensitive actuator that changes linear conformation in response to elevated tissue temperatures related to a developing device infection. The actuator can be in a linear configuration to change linear conformation as a direct function of constitutive material properties. In an alternative embodiment, the actuator is configured into a helical geometry to amplify the overall linear conformation changes of the constitutive materials comprising the actuator (i.e. helical actuators have increased linear translation when compared to linear actuators made of the same materials).

An embodiment includes a sensing cartridge that is inserted into the void space of a pedicle screw tulip.

An embodiment includes an orthopedic implant with a machined groove, through hole, or blind hole. The arc angle of the groove cross section includes material 185 to 360 degrees to contain a cartridge inserted into the groove or hole. Holes can be made with traditional machining bits or electrical discharge machining (EDM). Grooves can be machined with EDM or lollipop bits. The sensing cartridge has a radially expanding feature to interfere and bind with the groove. The cartridge can be included in the implant at the time of manufacture. Alternatively, the implant can include a multitude of grooves and or holes, and the clinician can insert instrumented cartridges in any number of these cartridge locations.

An embodiment includes a helical actuator that lies in a single plane and changes conformation in response to a biological signal correlated to implant infection. In one embodiment, the helical actuator is a shape memory polymer, shape memory alloy, LCE, bilayer of multiple materials with different thermomechanical properties and/or differential coefficients of thermal expansion, or combinations thereof. The helical actuator is terminated with a mobile radiopaque marker and the helical actuator changes conformation in response to increased tissue temperatures affiliated with implant infection. The helical actuator is contained in a disk shaped cartridge housing that has a static fiducial radiopaque marker to serve as a reference point. The cartridge housing and helical actuator are more radiolucent than the mobile and fixed radiopaque markers. Under x-ray imaging, the temperature driven change in actuator conformation can be detected and/or measured by observing the radial position of the mobile and fixed radiopaque markers.

An embodiment includes a cartridge anchoring within the cannulated screw channel or plate hole via a friction press fit.

An embodiment includes a cartridge anchoring within the cannulated screw channel or plate hole via screw or bayonet locking mechanism.

An embodiment includes a cartridge implantation that can be employed in conjunction with other implanted devices, or independent of other implants. For example, sensing cartridge can be affixed to other implanted devices (e.g., with sutures, medical adhesives), directly fixed to an anatomical feature (bone, ligament, muscle), or placed at the surgical site without fixating or connecting to other implant devices or other anatomical structures. For example, the sensing cartridge or envelope is implanted directly into a bone or surrounding tissue without the use of an additional fixation element.

An embodiment includes a temperature or other physical or chemical analyte sensitive actuator that lengthens or otherwise changes shape using a coefficient of thermal expansion (CTE). This includes bi-layers of materials with dissimilar CTEs shaped in spiral or helical conformations to achieve actuation and movement of a radiopaque marker. This includes shape memory alloys or other biocompatible metals. This includes semi-crystalline polymers. This includes LCEs.

An embodiment includes a temperature or other physical or chemical analyte sensitive actuator that lengthens or otherwise changes shape using polymeric materials. This includes bi-layers of materials with dissimilar CTEs shaped in a linear, spiral, or helical conformation to achieve actuation and movement of a radiopaque marker. This includes shape memory polymers. This includes semi-crystalline polymers. This includes liquid crystal elastomers.

An embodiment includes a sensor read out that is binary to detect infections.

An embodiment includes a sensor read out that is uni-directional such that, once actuated to indicate infection, the communication element permanently reads out a state indicating infection.

An embodiment includes a sensor read out that is reversible and indicative of the current state of the implant site. For example, a local increase in tissue temperature would stimulate a temperature sensitive sensing cartridge, but a decrease back to 37C body temperature would cause the actuator to reverse the conformation change and return to the original steady state configuration.

An embodiment includes a mechanism in which a marker is actuated bidirectionally through a multi-phase element in which the phase changes contribute to linear or radial translation of the marker. For example, a helically coiled LCE contributes a radial phase change in addition to a linear extension or contraction.

In an embodiment the sensing element contains a polymer that exhibits upper critical solution temperature or lower critical solution temperature capabilities. In one embodiment, this critical solution change is visible using ultrasound imaging. The sensing polymer is included in a sealed cartridge or applied as a polymeric coating to an implant surface. Alternatively, the polymer can be included in a window feature incorporated into the implanted medical device. For example the coating can be a web or membrane across a hole or slot in the implant. The echogenicity changing polymer fills the entire window, or may be included as a partial fill, with the remainder of the window volume filled with a positive control material with the same echogenicity as the sensing polymer at elevated temperature. For example, a square channel may be half filled with the sensing polymer, and half filled with a material that is not sensitive to infection analytes, but has a “positive control” echogenicity. Alternatively, a circular window may include an internal annulus of sensing polymer and the center of the annulus includes the polymer control (or vice versa). In this embodiment, a clinician could distinguish an infection via ultrasound imaging by the presentation of a partial (due to positive control) or full signal within the imaging window incorporated into the medical implant.

An embodiment includes a device system where multiple sensing modalities are employed to confirm infection. For example, an x-ray visible temperature sensor that changes conformation is used in conjunction with an ultrasound visible phase changing polymer that changes echogenicity in response to a physiologic parameter such as pH.

An embodiment includes a polymer that exhibits ordered to disordered phase transformations over a temperature range of 30-50 C

In an embodiment the cartridge interfaces with the torque transmitting features of an anchor, or drive transmitting features of an anchor.

In an embodiment fiducial markings on sensing elements, cartridges or inserts indicate the type of sensor that is implanted (e.g., temperature, chemical analyte).

An embodiment includes an infection sensing system comprising two or more implants. The first is integrated with the implanted medical device, and the additional implants are implanted contralaterally, or in a different anatomic location than the first implant. The additional implants serve as references to account for local implant biosignals (such as temperature) compared to whole-body biosignals. The additional implants can be subcutaneous for easy implantation and/or removal. The implant systems can have physical changes to biosignals, or generate digital signals. The multiple digital signals can be processed relative to each other to produce the infection diagnosis. Each implant can produce multiple signals associated with the local implant environment including temperature, reactive oxygen species produced by the local immune response, or other biologic factors secreted by bacteria associated with the implant infection. For example, tissue temperature recorded from multiple anatomic locations can discern elevated temperatures associated with local infection, systemic infection, and temperature increases associated with increased metabolic activity due to healing.

An embodiment includes an infection detection sensing system integrated with an implanted orthopedic medical device that consists of an integrated circuit powered via battery, RFID energy, or induction energy. The integrated circuit includes one or more sensors for infection related biosignals including temperature and/or physiologic analytes. Components of the circuit can be physically sealed and isolated from the biologic environment, while others can be in direct contact with tissues or interstitial fluids. The integrated circuit includes an antennae for wirelessly, non-invasively transmitting measured signals to a signal processing unit. The signal processing unit can simultaneously provide the wireless power to drive the integrated circuit, and record the biosignals. The signal processing unit can be handheld, or integrated with other diagnostic equipment in the orthopedic device workflow, including fluoroscopy imaging systems.

An embodiment includes an implant that changes physical properties in response to direct or indirect pathological changes that are associated with infections.

An embodiment includes a system for measuring physical changes in an implant that is exposed to an infection. Indirect methods of probing the implant to read the physical changes may be used, but contact methods including palpitation may also be used. Examples of indirect methods of probing for infection include using radiation, including any or multiple parts of the electromagnetic spectrum (e.g. optics, X-rays, infrared), acoustic (including ultrasound), thermal energy, or combinations of these (e.g. opto-acoustic).

An embodiment includes a coating, complete or partial, applied to current medical implants where the primary purpose of the coating is to change physical properties in the presence of an infection with little to no change in function of the implant.

An embodiment includes an implant or coating on an implant that contains two or more materials that have similar properties except for their response to one or more direct or indirect indicators of infection. For example, two areas of an implant are coated with similar materials except one of the materials changes properties (swelling, fluorescence intensity, phosphorescence lifetime, etc.) in response to temperature. The difference in response between the two areas provides a reference for improving the measurability of infection to the local environment of the implant.

An embodiment includes an implantable polymeric composition (whole, part, coating or partial coating of implant) that allows for empirical evaluation of pathologies through changes to the chemical orientation and/or conformation of the polymer backbone or functional pendant groups. In an embodiment, a polymer composition that is of implantable nature by itself or with the combination of positive and/or negative controls serves as an indicator that a pathology such as osteomyelitis is present in a patient.

Polymer compositions may include in whole, in-part, or any combination of polyethers, polyesters, polyorthoesters, polyamides, polyurethanes, polyureas, polyketones, polyphenols, polyolefins, polysiloxanes, polysaccharides, polyimides, polysulfides, polysulfones, polysulfoxides, polyamines, poly(meth)acrylates, polyacrylics, polycarbonates, and polyanhydrides. Additionally, the composition mentioned above may have a final composition that yields a thermoplastic or thermoset polymer.

Empirical evaluation encompasses changes in the geometry (2D and 3D), phosphorescence, fluorescence, glass transition temperature, melting temperature, density, radiopacity, stiffness, indirect conformational changes resulting in adjacent geometries.

Changes to chemical orientation encompass changes to fluorescent and phosphorescent moieties incorporated into the polymer backbone or as pendant groups. For example, IR808 as a fluorophore or sodium 4-styrenesulfonate as a phosphor can be incorporated into the polymer backbone and the functionality of the fluorescence and phosphorescence (modulated, through changes in emission wavelength or lifetime) through temperature, pH, oxidation, or combinations thereof. As an example of this embodiment, phosphorescence imaging is used as a non-invasive modality to assess the localized infection of an orthopedic implant using heat as the surrogate indicator of infection. Increased body temperature at the site of infection would cause the polymer system incorporated with the implant (either as a coating, polymer plug within a hole, coated/filled indention, other component, or combinations thereof) to change emission properties due to the rotational stability of the phosphor moiety. This change in wavelength emission provides a quantifiable signal for localized temperature. Further, a similar “control” portion of a similar polymer composition could be used as a clinical comparator. In this scenario, the control polymer has a similar emission wavelength as the analyte/temperature responsive polymer at 37.4° C., but the control polymer does not change phosphorescent characteristics in the presence of elevated temperature. Similarly, a “positive control” band of polymer could have a phosphorescence response that is similar to the temperature sensitive polymer at elevated temperature. By including negative and positive controls, the implant system provides the clinician with relative pass/fail criteria to assess the temperature responsive, infection indicating material.

An embodiment includes a polymer composition comprising 0.01%-10.00% of a fluorescent dye with emission wavelengths ranging from 780 to 2500 nm incorporated into a polymer matrix via a covalent bond. Example fluorescent dyes included molecules from the cyanine family. Fluorescent dyes for covalent incorporation into a polymer network include, but are not limited to indocyanine green (ICG) and carboxylic acid terminated ICG derivatives such as IR808, IR825, Cypate and the tertiary amine terminated IR800. Other fluorescent dyes include croconaine dyes. The polymer matrix comprises monomer repeat units that form covalent bonds with the dye, allowing 1) incorporation into the polymer backbone or 2) attachment to reactive pendant groups. Incorporation into the polymer backbone may be achieved through a hydroxyl reaction with an isocyanate group or amine (primary and secondary) with an isocyanate group through a polycondensation reaction. Examples of hydroxyl-terminated monomers for the polymer include: propylene glycols, butane diols, propane diols, glycerol, polyethylene glycols, polycarbonate glycols, polydioxane, polylactones, n-methyldiethanolamine, n-tert-butyldiethanolamine, or n-butyldiethanolamine, bis(2-hydroxypropyl)amine and 1,4-bis(2-hydroxyethyl)piperazine having a molecular weight between 100 Da to 5000 Da. Furthermore, examples of amine-terminated monomers include: diamino propanes, diamino butanes, ethylene diamine, PEG-Amine, PPG-Amine, PDMS-amine, and piperazine, having a molecular weight between 50 Da to 5000 Da. Other macromers can be added to the synthesis to impart other functional properties (e.g., controlling Tg, hydrophobicity, toughness, crystallinity, Tm, modulus, etc.).

Changes to chemical orientation also encompass changes in free volume of the polymer system that result in changes to the molecular and macro structure. For example, the polymer system may be composed of a mesogen, a chain extender, and a crosslinker to yield an LCE. An LCE can undergo a transition from a nematic phase to an isotropic phase as a result of external stimuli such as temperature, electromagnetic field, or solvation. In the process of the phase transformation conformational changes can be controlled to produce a specific geometry or applied stress. In an example, an LCE can be activated thermally at a pathologic temperature (>38° C.) to cause a change in geometry that is detectable under imaging modalities. Additionally, changes to the geometry could change the conformation of a radiopaque material, such as a nitinol wire, that possesses a first conformation but upon activation of the nitinol wire and/or the LCE, is switched to a second conformation that allows for evaluation of the presence of elevated temperature through standard radiographic techniques such as fluoroscopy, x-ray, and CT. Alternatively, the same polymer composition could utilize monomer groups such as N-isopropylacrylamide that result in polymers that exhibit changes in solubility based on temperature or pH known as lower temperature critical solution (LCST) networks. These networks are characterized by a change in intermolecular forces dominating to intramolecular forces dominating. Over this transition, a phase change is observed and similar to the LCE composition, can yield a change in geometry, or alternatively a change in opacity or echogenicity.

In an embodiment the polymer composition contains functional regions or zones that can respond from specific analytes associated with particular pathologies that elicit a measurable response. For example, a polymer composition may be tailored to detect infections associated with bone tissue by a reversible conformational change at both the micro and macro level. During an infection of bone tissue or osteomyelitis, the local temperature is increased as a result of a host response. Polymers in the proximity of this temperature change deviating from homeostasis (˜37.4° C.) to higher temperatures ranging from (38-42° C.) could undergo a pseudo phase transition or first order phase transition that allow for a geometry change to the polymer system that may allow for diagnostics.

In an embodiment, the polymer composition could have radiopaque or echogenic material dispersed in a fashion that creates a particular density responsive to stimuli or analytes for the diagnostics of pathologies. Upon a temperature change, conformational change of the polymer system may increase or decrease in volume and result in radiopacity/echogenicity decrease or increase, respectively. Further, the same polymer composition could have a metallic rod or continuous cylinder that is embedded into the material first geometry. Upon activation via stimuli or a specific analyte, the polymer system may change conformation and change the metallic component into a second geometry.

In another embodiment, the polymer system incorporates moieties that result in phase separation that is responsive to stimuli or analytes for the diagnostics of pathologies. For example, a polymer system such as a segmented polyurethane that has high degree of phase separation but switches to a low degree of phase separation as a result of temperature increase or interaction with a analyte such as uric acid would result in a change in echo or reflective properties using ultrasound as a diagnostic tool. As an example of this embodiment, ultrasound imaging is used as a non-invasive modality to assess the localized infection of an orthopedic implant using heat as the surrogate indicator of infection. Increased body temperature at the site of infection would cause the polymer system incorporated with the implant (either as a coating, polymer plug within a hole, coated/filled indention, other component, or combinations thereof) to change echogenic properties due to the degree of phase separation. This change in ultrasound reflection properties provides a quantifiable signal that is directly related to localized temperature. Further, a similar “control” portion of a similar polymer composition could be used as a clinical comparator. In this scenario, the control polymer has similar echogenicity as the analyte/temperature responsive polymer at 37° C., but the control polymer does not change ultrasound characteristics in the presence of elevated temperature. Similarly, a “positive control” band of polymer could have an echogenic response that is similar to the temperature sensitive polymer at elevated temperature. By including negative and positive controls, the implant system provides the clinician with relative pass/fail criteria to assess the temperature responsiveness, infection indicating material.

In yet another embodiment, the polymer compositions may change conformation through a chemical change to the polymer backbone or pendant group as a result of stimuli or analytes for the diagnostics of pathologies. Certain polymer systems may take advantage of the fenton reaction, haber-weiss, or reactions resulting in peroxyacids and undergo a reduction of their oxidative state. As a result, the polymer system would become more hydrophobic and a conformational change would occur. Additionally, the reduction of the system would result in the release of hydroxyl radicals (OH) that can act as agent to neutralize biologics such as bacteria. Other degradation products from a reduction reaction could include carbon dioxide gas, which could serve as an echogenic indicator of the reaction. This gas could be trapped as bubbles in the polymer system for long timescale observation using ultrasound imaging.

Examples are now addressed.

Example 1. A system comprising: (a) a medical implant comprising at least one of a bone anchor, a hip implant, a knee implant, a should implant, a plate, or combinations thereof; (b) a sealed housing coupled to the medical implant; (c) a liquid crystal elastomer (LCE) included in the sealed housing; wherein the LCE is configured to transition from a nematic phase to an isotropic phase in response to exposure of the LCE to a temperature above 38 degrees Celsius.

For example, the sealed housing may be included in or attached to the medical implant. Further, in other embodiments the LCE may be programmed to transition from a nematic phase to an isotropic phase in response to exposure of the LCE to a temperature above 36, 37, 39, 41, 43 degrees Celsius or more.

Example 2. The system of Example 1, wherein the LCE is configured to change its shape in response to the LCE transitioning from the nematic phase to the isotropic phase.

Example 3. The system according to any of Examples 1-2, wherein the LCE includes a mesogen, a chain extender, and a crosslinker.

As addressed in U.S. Pat. No. 11,780,154, LCEs are a class of stimuli responsive polymers that undergo large, reversible, anisotropic shape change in response to a variety of stimuli, including heat and light. Unlike many materials that undergo reversible shape change, these materials neither require an external load nor an aqueous environment, making them ideal candidates for many applications. For LCEs to undergo reversible shape-change in the absence of load, the LCE should be crosslinked in an aligned state. Commonly, partially-crosslinked LCEs are fully crosslinked under a mechanical load leading to permanent orientation of the liquid crystal (LC) molecules within the polymer network. On heating, the resulting aligned LCEs contract along the alignment direction, or nematic director, and expand in the perpendicular axes. Other programming methods are described in U.S. Pat. No. 11,780,154.

While in many cases herein an LCE is used as an actuator, in other embodiments the following materials may be used as temperature or environment-sensitive actuators: shape memory polymer, shape memory alloy, bilayer of multiple materials with different thermomechanical properties and/or differential coefficients of thermal expansion, or combinations thereof.

Example 4. The system according to any of Examples 1-3 comprising a first radiopaque marker coupled to the LCE.

Example 5. The system of Example 4, wherein the first radiopaque marker is configured to physically move in response to the LCE transitioning from the nematic phase to the isotropic phase.

Example 5.1 The system according to any of Examples 5-5.1 comprising a second radiopaque marker fixedly coupled to the sealed housing.

For example, see FIG. 1(a).

Example 5.2 The system according to Example 5.1, wherein the first radiopaque marker is configured to physically move with respect to the second radiopaque marker in response to the LCE transitioning from the nematic phase to the isotropic phase.

5.3 The system of Example 5, wherein: (a) the first radiopaque marker is configured to physically move a first distance in response to exposure of the LCE to the temperature above 38 degrees Celsius; (b) the first radiopaque marker is configured to physically move a second first in response to exposure of the LCE to the temperature above 39 degrees Celsius.

Example 5.4 The system of Example 5, wherein: (a) the first radiopaque marker is configured to physically move a first distance in response to exposure of the LCE to a temperature between 0 and 38 degrees Celsius; (b) the first radiopaque marker is configured to physically move a second first in response to exposure of the LCE to the temperature above 38 degrees Celsius.

Example 6. The system according to any of Examples 1-5.1, wherein the LCE is coiled in the nematic phase.

Example 6.1 The system of Example 6, wherein: (a) the first radiopaque marker includes a long axis and a short axis; (b) the first radiopaque marker is configured to rotate about the short axis in response to the LCE transitioning from the nematic phase to the isotropic phase.

Example 6.2 The system of Example 6, wherein the first radiopaque marker is configured to rotate about an axis in response to the LCE transitioning from the nematic phase to the isotropic phase.

For example, see FIGS. 5(a)-(d) and/or FIGS. 6(a)-(d). See, for example, axis 522 in FIG. 5(a) or axis 622 in FIG. 6(a).

Example 6.3 The system of Example 6.2, wherein the LCE is disposed in a plane and is configured to rotate within the plane in response to the LCE transitioning from the nematic phase to the isotropic phase.

Example 7. The system according to any of Examples 1-6, wherein the LCE includes a polymer that comprises polymerized monomers, the monomers including RM257 (CAS 174063-87-7), R6M/RM82 (CAS 125248-71-7), RM23 (CAS 83847-14-7), RM105 (CAS 82200-53-1), 1,4-butanediol diacrylate, Poly(ethylene glycol) diacrylate, Ethylene glycol diacrylate, Tri(ethyleneglycol) diacrylate, Tetra(ethylene glycol) diacrylate, 1,3-butanediol diacrylate, Neopentyl glycol diacrylate, Tritolylamine diacrylate, 1,3-Butanediol dimethacrylate, Ethylene glycol dimethacrylate, Triethylene glycol dimethacrylate, 1,6-Hexanediol dimethacrylate, 1,4-Butanediol dimethacrylate, Diurethane dimethacrylate, 1,10-Decanediol dimethacrylate, Bis(2-methacryloyl)oxyethyl disulfide, 2-Methyl-1,3-butadiene, 1,4 Pentadiene, 1,5-Hexadiene, 1,3,4-Thiadiazole-2,5-dithiol, Toluene-3,4-dithiol, 1,6-Hexanedithiol, 2,2′-(ethylenedioxy)dithiol, Tetra(ethylene glycol)dithiol, p-Terpheyl-4-4″-dithiol, 5-vinyl-2-norbornene, pentaerythritol tetrakis(3-mercaptopropionate), Trimethylolpropane tris(3-mercaptopropionate), Trimethylolpropane trimethacrylate, 1,3,5-Triallyl-1,3,5-triazine-2,4,6(1H,3H,5H)-trione, 2,4,6-Triallyloxy-1,3,5-triazine, Tris[2-(acryloyloxy)ethyl] isocyanurate, Pentaerythritol tetraacrylate, or combinations thereof.

LCEs may include a composition having reactive mesogens (RMs) that are polymerizable via radical polymerization and click chemistry mechanisms such as radical initiated thiol-ene click chemistry or ebeam radiation crosslinking. Example RMs include RM257 (CAS 174063-87-7), R6M/RM82 (CAS 125248-71-7), RM23 (CAS 83847-14-7), RM105 (CAS 82200-53-1), or combinations thereof. These mesogen monomers can be reacted with difunctional monomers with thiol, acrylate, methacrylate, norbornene, or alkene terminal groups to yield thermoplastic polymers. Example monomers include 1,4-butanediol diacrylate, Poly(ethylene glycol) diacrylate, Ethylene glycol diacrylate, Tri(ethyleneglycol) diacrylate, Tetra(ethylene glycol) diacrylate, 1,3-butanediol diacrylate, Neopentyl glycol diacrylate, Tritolylamine diacrylate, 1,3-Butanediol dimethacrylate, Ethylene glycol dimethacrylate, Triethylene glycol dimethacrylate, 1,6-Hexanediol dimethacrylate, 1,4-Butanediol dimethacrylate, Diurethane dimethacrylate, 1,10-Decanediol dimethacrylate, Bis(2-methacryloyl)oxyethyl disulfide, 2-Methyl-1,3-butadiene, 1,4 Pentadiene, 1,5-Hexadiene, 1,3,4-Thiadiazole-2,5-dithiol, Toluene-3,4-dithiol, 1,6-Hexanedithiol, 2,2′-(ethylenedioxy)dithiol, Tetra(ethylene glycol)dithiol, p-Terpheyl-4-4″-dithiol, 5-vinyl-2-norbornene, and mixtures thereof. Other embodiments may react the mesogen monomers with polyfunctional monomers (Fn>2) to yield thermoset LCE's. Example monomers include pentaerythritol tetrakis(3-mercaptopropionate), Trimethylolpropane tris(3-mercaptopropionate), Trimethylolpropane trimethacrylate, 1,3,5-Triallyl-1,3,5-triazine-2,4,6(1H,3H,5H)-trione, 2,4,6-Triallyloxy-1,3,5-triazine, Tris[2-(acryloyloxy)ethyl]isocyanurate, Pentaerythritol tetraacrylate.

8. The system according to any of Examples 1-7 wherein the LCE is configured to transition from the isotropic phase to the nematic phase in response to exposure of the LCE to a temperature below 38 degrees Celsius.

9. The system according to any of Examples 1-8, wherein: (a) the LCE has a first shape in the nematic phase; (b) the LCE has a second shape in the isotrophic phase; (c) the LCE is configured to transition from the first shape to the second shape in response to the LCE transitioning from the nematic phase to the isotropic phase; (d) the LCE is configured to transition from the second shape to the first shape in response to the LCE transitioning from the isotropic phase to the nematic phase.

Example 10. The system of Example 9 wherein the LCE is configured to transition from the second shape to the first shape after already having transitioned from the first shape to the second shape.

Example 11. The system according to any of Examples 1-10, wherein: the LCE includes first and second ends that oppose one another; the first end is fixedly coupled to the sealed housing; the second end is not fixedly coupled to the sealed housing.

For example, see FIG. 1.

Example 12. The system according to any of Examples 1-11 wherein the sealed housing includes a liquid.

For example, the liquid may help transfer thermal energy to the LCE.

Example 13. The system of Example 12, wherein the liquid has a thermal conductivity (W/m-K) between 0.1 and 0.7.

For example, the liquid may include any of the following: ethylene glycol (0.252 W/m-K), deionized water (0.603 W/m-K), mineral oil (0.126 W/m-K), Envirotemp FR3 natural ester (0.167 W/m-K), Midel 7131 (0.144 W/m-K), or combinations thereof.

Example 14. The system of Example 13, wherein the liquid includes at least one of water, a semi-polar solvent, a non-polar solvents, or combinations thereof.

Example 15. The system according to any of Examples 1-11 wherein the sealed housing includes a gas.

For example, the gas may help transfer thermal energy to the LCE.

Example 16. The system of Example 15, wherein the gas has a thermal conductivity (W/m-K) between 0.01 and 0.03.

For example, the as may include dry air (0.026 W/m-K), Argon (0.016 W/m-K), Carbon dioxide (0.0146 W/m-K), Nitrogen (0.024 W/m-K).

Example 17. The system of Example 16, wherein the gas includes at least one of ambient air, carbon dioxide, nitrogen, argon, or combinations thereof.

Example 18. The system according to any of Examples 1-17, wherein: (a) the sealed housing includes an outer wall including a material; (b) the medical implant includes a void with a wall, the wall of the void including the material; (c) the void is sized to receive the sealed housing.

For example, the both the housing outer wall and the implant void's wall may both include titanium to prevent or lessen any chemical reaction between the housing and implant due to walls having dissimilar materials.

For example, the medical device may include an orthopedic implant with several similarly sized voids. The voids may be able to accommodate a bone anchor, the sealed housing, or a bone anchor having the sealed housing.

For example, see FIG. 8(c).

Example 19. The system according to any of Examples 1-18, wherein the LCE has a negative coefficient of thermal expansion.

A linear translation in response to physical analytes that moves a readable marker with a translation range between 0.1 mm and 100 mm corresponding to the range of interest in the analyte. For example, an actuator with an initial length of 20 mm that elongates (or shortens) between 0-5 mm indicates a temperature of 37-45 C. In one embodiment, a length of 20 mm indicates 37 C and 25 mm indicates 45 C. In another embodiment, a length of 20 mm indicates 45 C and a 25 mm length indicates 37 C. A second example is an actuator with an initial length of 10 mm that has a shortening range of 0-1 mm over the same temperature range. In the second example, a length of 10 mm indicates 37 C and 9 mm indicates 45 C.

Example 20. The system according to any of Examples 1-19, wherein: the medical implant is a bone anchor; the sealed housing is located in a proximal half of the bone anchor; the distal half of the bone anchor is configured to implant into bone before the proximal half of the bone anchor.

Example 21. The system of Example 6, wherein: the sealed housing is not completely enclosed by the medical device; a portion of the sealed housing is configured to interface tissue or bodily fluids after final implantation of the medical device into a patient.

Example 22. The system of Example 21, wherein the sealed housing is not monolithic with the medical device.

For example, the sealed housing does not share a wall of the implant that was milled or 3-D printed along with the rest of the, for example, hip implant.

Example 23. The system of clam 21, wherein the sealed housing is coupled to the medical device via at least one of threads, an adhesive, a weld, or combinations thereof.

Example 24. A method comprising imaging the medical implant according to any of Examples 1-23.

Example 25. The method of Example 24 comprising detecting a change in shape of the LCE.

Example 26. The method of Example 24 comprising detecting the physical movement of the LCE.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

What is claimed is:

1. A system comprising:
a medical implant comprising at least one of a bone anchor, a hip implant, a knee implant, a shoulder implant, an orthopedic plate, or combinations thereof;
a sealed housing coupled to the medical implant;
a liquid crystal elastomer (LCE) included in the sealed housing;
wherein the LCE is configured to transition from a nematic phase to an isotropic phase in response to exposure of the LCE to a temperature above 38 degrees Celsius.

2. The system of claim 1, wherein the LCE is configured to change its shape in response to the LCE transitioning from the nematic phase to the isotropic phase.

3. The system of claim 1, wherein the LCE includes a mesogen, a chain extender, and a crosslinker.

4. The system of claim 3 comprising a first radiopaque marker coupled to the LCE.

5. The system of claim 4, wherein the first radiopaque marker is configured to physically move in response to the LCE transitioning from the nematic phase to the isotropic phase.

6. The system of claim 5 comprising a second radiopaque marker fixedly coupled to the sealed housing, wherein the first radiopaque marker is configured to physically move with respect to the second radiopaque marker in response to the LCE transitioning from the nematic phase to the isotropic phase.

7. The system of claim 6, wherein:
the first radiopaque marker is configured to physically move a first distance in response to exposure of the LCE to the temperature above 38 degrees Celsius;
the first radiopaque marker is configured to physically move a second distance in response to exposure of the LCE to the temperature above 39 degrees Celsius.

8. The system claim 6, wherein the LCE is coiled in the nematic phase.

9. The system of claim 8, wherein the first radiopaque marker is configured to rotate about an axis in response to the LCE transitioning from the nematic phase to the isotropic phase.

10. The system of claim 6, wherein the LCE is disposed in a plane and is configured to rotate within the plane in response to the LCE transitioning from the nematic phase to the isotropic phase.

11. The system of claim 10, wherein the LCE is coiled in the nematic phase.

12. The system of claim 11, wherein:
the medical implant includes a plate having a void;
the sealed housing is configured to fit within the void.

13. The system of claim 6, wherein:
the LCE has a first shape in the nematic phase;
the LCE has a second shape in the isotropic phase;
the LCE is configured to transition from the first shape to the second shape in response to the LCE transitioning from the nematic phase to the isotropic phase;
the LCE is configured to transition from the second shape to the first shape in response to the LCE transitioning from the isotropic phase to the nematic phase.

14. The system of claim 13, wherein the LCE is configured to transition from the second shape to the first shape after already having transitioned from the first shape to the second shape.

15. The system of claim 6, wherein:
the LCE includes first and second ends that oppose one another;
the first end is fixedly coupled to the sealed housing;
the second end is not fixedly coupled to the sealed housing.

16. The system of claim 15, wherein the sealed housing includes a liquid.

17. The system of claim 6, wherein:
the medical implant is a bone anchor;
the sealed housing is located in a proximal half of the bone anchor;
a distal half of the bone anchor is configured to implant into bone before the proximal half of the bone anchor.

18. The system of claim 6, wherein:
the sealed housing is not completely enclosed by the medical implant;
a portion of the sealed housing is configured to interface tissue or bodily fluids after final implantation of the medical implant into a patient.

19. The system of claim 18, wherein the sealed housing is not monolithic with the medical implant.

20. The system of claim 18, wherein the sealed housing is coupled to the medical implant via at least one of threads, an adhesive, a weld, or combinations thereof.
